(19) United States
(12) Patent Application Publication
(10) Pub. No.: US 2023/0124158 A1
(43) Pub. Date: Apr. 20, 2023

(54) ASSESSING WALKING STEADINESS OF MOBILE DEVICE USER

(71) Applicant: Apple Inc., Cupertino, CA (US)

(72) Inventors: Mariah W. Whitmore, Cupertino, CA (US); Jaehyun Bae, San Carlos, CA (US); Richard A. Fineman, Campbell, CA (US); Sheena Sharma, Carbondale, IL (US); Asif Khalak, Belmont, CA (US); Adeeti V. Ullal, Emerald Hills, CA (US)

(21) Appl. No.: 17/832,569

(22) Filed: Jun. 3, 2022

Related U.S. Application Data
(60) Provisional application No. 63/197,327, filed on Jun. 4, 2021.

(51) Int. Cl. A61B 5/11; A61B 5/00

(52) U.S. Cl. CPC A61B 5/112; A61B 5/6898; A61B 5/7246; A61B 5/7267; A61B 5/7275; A61B 5/1117; A61B 2562/0219

(57) ABSTRACT

Embodiments are disclosed for assessing walking steadiness of a mobile device user. In some embodiments, a method comprises: obtaining, with at least one processor of a mobile device, one or more mobility metrics indicative of a user's mobility, the mobility metrics obtained at least in part from a time series of sensor data output by at least one sensor of the mobile device; evaluating, with the at least one processor, the one or more mobility metrics over one or more specified time periods to derive one or more longitudinal features indicative of variability of the user's gait; and generating, with the at least one processor, at least one walking steadiness indicator for the user based on one or more walking steadiness component models and the one or more longitudinal features. Also disclosed are embodiments for training the component models.

### CROSS-RELATED APPLICATIONS

This application claims the benefit of priority from U.S. Provisional Patent Application No. 63/197,327, for “Walking Steadiness and Fall Risk Assessment,” filed on Jun. 4, 2021, which patent application is incorporated by reference herein in its entirety.

### TECHNICAL FIELD

This disclosure relates generally to health monitoring, and in particular walking steadiness assessment.

### BACKGROUND

Every year, nearly 1 in 3 adults over the age of 65 report falling. Fall death rates are on the rise, increasing 3% per year from 2007 to 2016 among adults 65 and older. The approximate cost of a fall treated in the emergency department, hospital, or outpatient setting is $10,000, and with the aging baby boomer population, it is likely that falls will become an even greater health concern and economic burden. There are evidence-based strategies that have been proven to reduce falls, if individuals can be made aware they have high fall risk and take appropriate action.

Fall risk is most commonly evaluated in a doctor's office with a patient filling out a questionnaire and potentially doing a short functional assessment such as a Timed Up and Go (TUG) test. The questionnaire asks about a variety of risk factors including prior history of falls, intrinsic mobility limitations, medication usage and depression. All of the reported factors are added up, and if the sum is above a certain threshold, the patient has a high fall risk and follow-up education and interventions are discussed with the patient. The TUG test is a simple test of overall functional mobility, where the patient is asked to stand up from a chair, walk 10 meters, turn around, walk back, and sit back down. This procedure is timed and above a certain time is indicative that the patient may be at increased risk for falls. The completion of these assessments is dependent on integration of fall prevention in primary care practices, which is low and only routinely done for adults 65 years and older.

### SUMMARY

Embodiments are disclosed for assessing walking steadiness of a mobile device user.

In some embodiments, a method comprises: obtaining, with at least one processor of a mobile device, one or more mobility metrics indicative of a user's mobility, the mobility metrics obtained at least in part from a time series of sensor data output by at least one sensor of the mobile device; evaluating, with the at least one processor, the one or more mobility metrics over one or more specified time periods to derive one or more longitudinal features indicative of variability of the user's gait; and generating, with the at least one processor, at least one walking steadiness indicator for the user based on a gait compensatory model and the one or more longitudinal features.

In some embodiments, the at least one walking steadiness indicator is an entropy value computed over the one or more specified time periods.

In some embodiments, the at least one walking steadiness indicator is a degree of dispersion computed over the one or more specified time periods, and the dispersion value computed by: determining, with the at least one processor, metrics for step length, walking speed and cadence of the user based on the sensor data; determining, with the at least one processor, correlations between the metrics over the one or more specified time periods; and determining, with the at least one processor, the degree of dispersion based on the determined correlations.

In some embodiments, the degree of dispersion is determined based on parameters of an ellipse-fitted Poincaré plot that indicate at least one of short-term or long-term variability in the user's gait.

In some embodiments, the method further comprises: determining, with the at least one processor and prior to the evaluating, that the sensor data covers a duration that satisfies a minimum threshold time.

In some embodiments, the method further comprises: generating, with the at least one processor, the at least one walking steadiness indicator based on the gait compensatory model, the one or more longitudinal features and population norms.

In some embodiments, the population norms are represented as percentiles by age.

In some embodiments, a method comprises: obtaining, with at least one processor of a mobile device, one or more mobility metrics indicative of a user's mobility, the mobility metrics obtained at least in part from a time series of sensor data output by at least one sensor of the mobile device; evaluating, with the at least one processor, the one or more mobility metrics over one or more specified time periods to derive one or more longitudinal features indicative of sustained walking; and generating, with the at least one processor, at least one walking steadiness indicator for the user based on a capacity model and the one or more longitudinal features.

In some embodiments, generating the at least one walking steadiness indicator based on the capacity model and the one or more longitudinal features, further comprises: estimating vertical acceleration from the sensor data; estimated walking speed from the vertical acceleration; estimating cadence and step length of the user for each step cycle based on the sensor data; generating the at least one walking steadiness indicator for the user based on the estimates of user walking speed, step length and cadence.

In some embodiments, generating the at least one the walking steadiness indicator based on the capacity model and the one or more longitudinal features, further comprises: estimating periods of continuous stepping based on the sensor data; detecting periods of consecutive stepping based on the sensor data; grouping the periods into time periods of specified durations; and evaluating features for particular time periods to generate the at least one walking steadiness indicator.

In some embodiments, the features include at least one of: cadence, step length, double step time, median speed over a specified time duration or speed coefficient of variance.

In some embodiments, the method further comprises: generating, with the at least one processor, the at least one walking steadiness indicator based on the capacity model, the one or more longitudinal features and population norms.

In some embodiments, a method comprises: obtaining, with at least one processor of a mobile device, one or more mobility metrics indicative of a user's mobility, the mobility metrics obtained at least in part from a time series of sensor data output by at least one sensor of the mobile device; evaluating, with the at least one processor, the one or more mobility metrics over one or more specified time periods to derive one or more longitudinal features indicative of frequency characteristics of the user's walking pattern; and generating, with the at least one processor, at least one walking steadiness indicator for the user based on a gait smoothness model and the one or more longitudinal features.

In some embodiments, the gait smoothness model calculates gait smoothness using an index of harmonicity to determine closeness of the user's gait to a sine wave at a dominant frequency of the user's walking.

In some embodiments, the smoothness is calculated for multiple directions of the user's motion.

In some embodiments, the method further comprises: generating, with the at least one processor, the at least one walking steadiness indicator based on the gait smoothness model, the one or more longitudinal features and population norms.

In some embodiments, a method comprises: obtaining, with at least one processor of a mobile device, training data indicative of mobility limitations; and training, with the at least one processor and using the training data, a plurality of machine learning models for estimating walking steadiness, wherein the plurality of component models are trained to different targets.

In some embodiments, the training data includes weighted questions for fall risk discrimination that are consistent predictors of mobility limitations.

In some embodiments, the questions are Cox-weighted.

In some embodiments, different Cox-weights are based on age.

In some embodiments, the training data includes at least one of functional gait assessments or functional status questionnaires.

In some embodiments, training the plurality of machine learning models for estimating walking steadiness includes training the machine learning models to their own residuals.

In some embodiments, the residuals are trained against a plurality of the machine learning models versus a null model.

Other embodiments can include an apparatus, computing device and non-transitory, computer-readable storage medium.

Particular embodiments described herein provide one or more of the following advantages. A mobile device platform (e.g., a smartphone, smartwatch, fitness band) is disclosed that combines sensors, such as accelerometer, gyroscope, magnetometer, barometer, and Global Navigation Satellite System (GNSS) (e.g., Global Position System (GPS)), to assess the quality and dynamic balance of a user's walking gait (referred to hereinafter as “walking steadiness”), classifies how the user's walking steadiness compares to the walking steadiness of the general population, and predicts a likelihood of the user falling (“fall risk”) in the future (e.g., the next 12 months).

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1A is a block diagram of a system for assessing walking steadiness and fall risk, according to some embodiments.

FIG. 1B illustrates compensatory walking patterns, according to some embodiments.

FIG. 1C illustrates multi-directional gait smoothness, according to some embodiments.

FIG. 2A illustrates a basic approach for training feature weights for component models, according to some embodiments.

FIG. 2B illustrates training feature weights for component models using stacked models to train to their own residuals, according to some embodiments.

FIG. 2C illustrates training feature weights for component models using stacked models to train a residual against many models versus a null model to only train for gains, according to some embodiments.

FIGS. 2D-1 and 2D-2 illustrate training feature weights for component models using an enhanced age balancing approach, according to some embodiments.

FIG. 3 illustrates a training architecture using an ensemble of machine learning models, according to some embodiments.

FIG. 4 is a flow diagram of a process of assessing walking steadiness and fall risk, according to some embodiments.

FIG. 5 is example system architecture implementing the features and operations described in reference to FIGS. 1-4.

### DETAILED DESCRIPTION

In an embodiment, several components are required to estimate and classify the quality of a user's walking steadiness and subsequently infer their prospective risk of falling. To estimate walking steadiness requires component biomechanical models of steadiness, measures of behavior, and longitudinal evaluation over different timescales. All of the individual biomechanical component models used to estimate steadiness are combined or fused to create a single walking steadiness indicator that informs functional mobility of the user across a wide range of mobility levels and limitations. When assessed at scale across a general population with a wide range of ages, functional status, and prior fall history, the steadiness indicator can be classified into particular categories. The category in which a user is classified indicates they may be at relatively increased likelihood of having mobility risk factors or future risk of falling.

### Example System

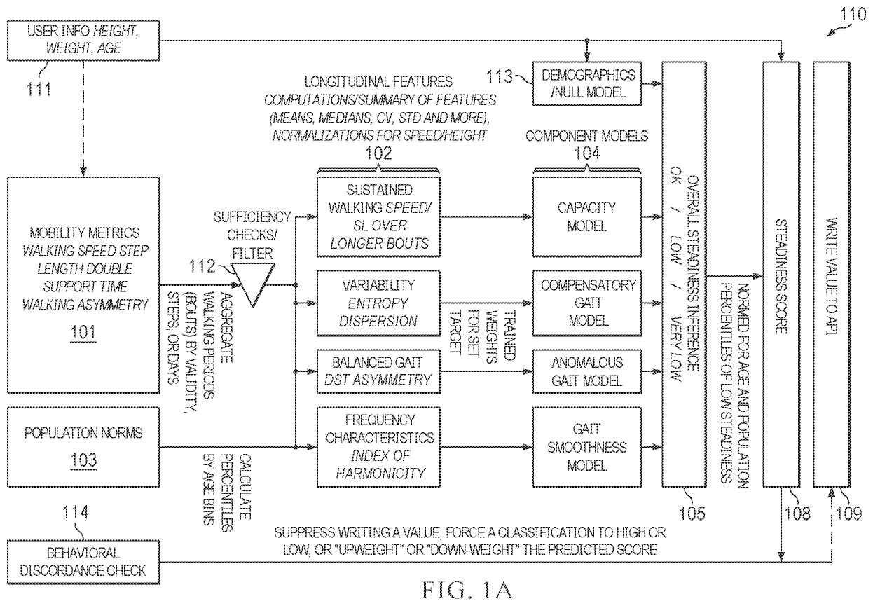

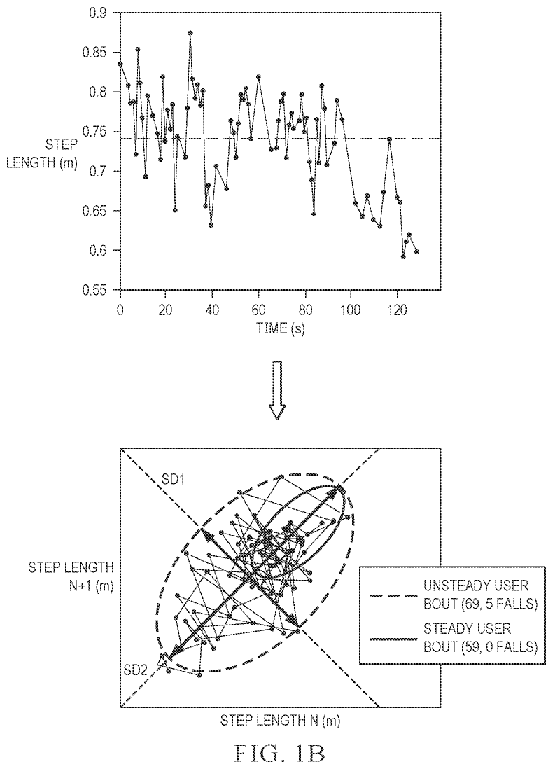

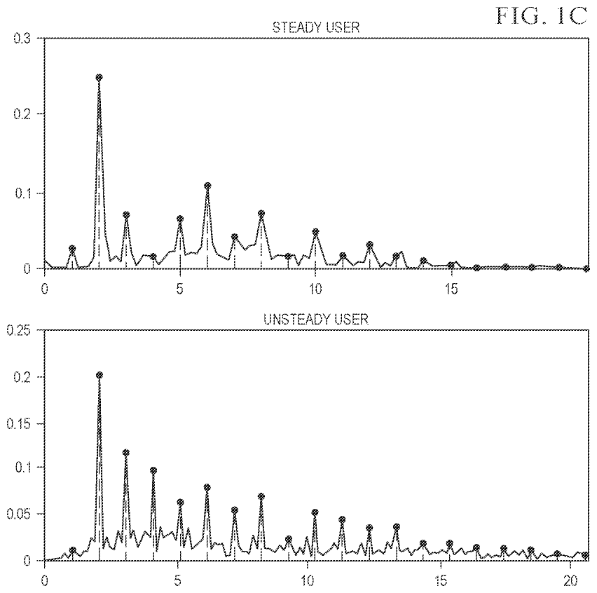

FIG. 1A is a block diagram of a system 100 for assessing walking steadiness and fall risk, according to some embodiments. System 100 includes user information 111 (e.g., height, weight, age), mobility metrics 101, longitudinal features 102, population norms 103, walking steadiness component models 104, fused inference model 105 and behavioral discordance checker 114. The output of fused inference model 105 is steadiness score 108, which can be written to, e.g., an application programming interface (API) 109 of a health monitoring and/or fitness framework (e.g., Apple Inc.'s HealthKit™) for a mobile device or other device, which provides access the fall risk score to various applications (e.g., fitness applications, health monitoring applications).

### I. Mobility Metrics

In an embodiment, mobility metrics 101 can include but are not limited to: walking speed, step/stride length, double support time (DST) and walking asymmetry. Mobility metrics 101 can be determined from sensor data, such as acceleration data and/or rotation rate data obtained from accelerometer and gyro sensors, respectively, which are embedded or coupled to a mobile device worn by a user (e.g., a smartwatch, smartphone).

In some embodiments, walking speed can be determined using equations of motion applied to acceleration data, and/or can be estimated by a localization system embedded in or coupled to the mobile device, such as a Global Navigation Satellite System (GNSS) receiver (e.g., GPS receiver) and/or wireless network localization system (e.g., cellular network positioning system, WIFI localization, etc.).

Step length is the distance between the user's feet when the user takes a step. The longer the step length the better indicator of changes in strength and coordination. In some embodiments, step length can be determined based on acceleration data in the time domain or frequency domain to count steps. In some embodiments, step length is estimated using a machine learning model.

DST occurs when both feet are in contact with the ground simultaneously. DST is measured as a percentage of the user's overall walking time. A lower percentage means the user is spending more time on one foot, which is an indicator of strength and balance. During a typical walk, the DST measure will fall between 20% and 40%. In some embodiments, DST is the sum of the times elapsed during two periods of double support in a gait cycle based on, e.g., step counts for right/left legs or other data.

Walking asymmetry occurs when the user favors one foot over the other while walking. A lower percentage is healthier and 0% means balanced walking. A higher walking asymmetry reading could be the result of a sports injury, disease or surgery. Some degree of asymmetry might be normal, if, for example, the user's legs are different lengths. In some embodiments, walking asymmetry can be determined, e.g., based on patterns in rotation or rotation rate data in a two-dimensional (2D) or three-dimensional (3D) reference frame.

In some embodiments, an input can be the user's height. The user's height provides more accurate estimates of speed and other gait metrics. User height can also be used to normalize gait parameters. For example, a tall, unsteady user may walk faster or with longer steps than a specified population of users. The user's height can be used to normalize the user's gait parameters to the specified population of users.

### II. User Information

In some embodiments, user information 111 is input by the user through a graphical user interface of the mobile device. In some embodiments, height can be entered by the user through the user interface or estimated based on an estimated stride length determined from sensor data. A high body mass index (BMI) affects gait parameters because of extra loading on joints and lower limb muscles. In particular, BMI can affect the length of time the user requires to be supported by both feet (e.g., DST). The user's weight can be used to retrain the accelerometer and gyroscope-based features of gait cycles to attain higher accuracy. Notions of steadiness and capacity differ by age. Age can be used to normalize and select an appropriate model to give steadiness classification to the user.

### III. Longitudinal Features

In some embodiments, prior to computing derived longitudinal features 102, mobility metrics 101 are aggregated over time. Different periods of walking provide different information. For example, continuous walking periods can be aggregated using step counts. Another example would be aggregating by time. By aggregating mobility metrics 101 over longer time windows, variations of typical gait parameters can be more robustly calculated (e.g., over last 7 days, or even a whole day or last 28 days).

Longitudinal features 102 can be derived from mobility metrics 101 that provide indications of walking steadiness level, either good steadiness or poor steadiness, at many different time scales (e.g., hours, days, weeks, months, years). In some embodiments, mobility metrics 101 are evaluated over every walking cycle for an assessment of “in-the-moment” walking quality, where identified moments of imbalance can be accumulated throughout the day and beyond. Mobility metrics 101 can be evaluated over the course of a week to account for day-of-the-week fluctuations largely driven by behavioral and activity level differences between the normal work-week and weekends. Mobility metrics 101 can be evaluated over the course of months and years to observe potentially expected gradual decline (e.g. due to natural aging) or gradual improvements (e.g., during adoption of a particular workout routine). All of these timescales can help inform walking steadiness for a given point in time by taking into account both current and past mobility metrics 101.

### IV. Population Norms

In some embodiments, population norms 103 are determined from mobility metrics 101 and indicate normative behavior for different user populations based on demographics, such as gender, age and group strata (e.g., users with disabilities). For example, population norms can be expressed as percentiles (e.g., 95^{th} percentile) over age bins.

### V. Sufficiency Checker/Filter

In some embodiments, data sufficiency checks/filter 112 are applied to the mobility metrics 101 to ensure sufficient data to determine walking quality or steadiness. For example, a check can be whether a user has walked a sufficiently long bout (continuous walking segment) to capture walking metrics that are not impacted by accelerations and/or decelerations and that predominate short bouts (e.g., at least ˜1 minute). Another example would be whether the user has provided a variety of estimates over a specified time period (e.g., 7 days). Yet another example would be has the user provided enough long-term history to ensure outlier behavior for the specified time period due to vacation or otherwise are excluded (e.g., 30+ bouts in 28 days), e.g., a minimum 2 week observation period to start, followed by rolling time windows.

### VII. Behavioral Discordance Checker

In some embodiments, a user may provide key signals to system 100 other than walking that can help correct, adjust or suppress the steadiness classification score. Behavioral discordance checker 114 is configured to suppress the reporting of steadiness indicator 108, forcing steadiness indicator 108 to high or low risk, or increase/decrease the steadiness indicator 108. For example, key signals can include workout information from a smartwatch or other device that indicates the user's running gait and/or walking speed, workout information through user logged information via a mobile application (e.g., strength training, mapmyrun routes, etc.), or signals that indicate a user's routine, such as commonly visited locations used to determine where the user goes to the gym frequently though rarely carries a phone.

### VIII. Walking Steadiness Component Models

Walking steadiness is a holistic concept used to assess overall walking quality. There are many different physiological contributions to walking quality that can each be modeled independently to enable identification of low or poor steadiness across a wide range of mobility levels and types of limitations. In some embodiments, walking steadiness component models 104 include but are not limited to: capacity (e.g., how fast the user can walk, how long of steps the user takes, how long can the user walk in challenging environments), gait compensatory component model (e.g. how frequently does the user take corrective steps leading to high variability in walking parameters) and balanced gait component model (e.g., how much time is spent by the user with both feet on the ground, do both feet have similar walking parameters, how much side-to-side motion occurs while the user is walking, behaviors by the user that are indicative of balance issues, etc.). Each component model is described below in turn.

i. Capacity (Strength/Endurance) Model

The speed of walking and length of strides maximally achieved and their product are indicators of strength, which is an indicator of walking steadiness. In some embodiments, the capacity component model fuses inertial sensor signals to estimate vertical acceleration. From the vertical acceleration, walking speed is estimated using an inverted pendulum model. From the vertical acceleration, each step cycle is segmented to determine cadence and to derive step length. The estimates of speed, step length and cadence are then combined to derive capacity indicators, which are output by the capacity model An example capacity indicator is N % walking speed (e.g., N=96) divided by step length per bout.

The duration of continuous walking typically completed, and the speed and cadence achieved for those durations are indicators of endurance, which is an indicator of walking steadiness. In some embodiments, the capacity model fuses inertial sensor signals to estimate periods of continuous stepping and detect periods of consecutive stepping (i.e., sustained walking) and group those periods into bouts of particular durations and evaluate features for particular bout lengths to evaluate indications of endurance. Some examples of endurance features include but are not limited to at least one of: cadence, step length, DST and speed medians during bouts 1-N minutes (e.g., N=10) long or any subset of similar time durations, and cadence, step length, DST and a speed coefficient of variance during bouts 1-N minutes long or any subset of similar time durations and bout durations.

ii. Gait Compensatory (Variability) Model

Variability in walking characteristics evaluated at different time scales, including dispersion over a specified time period (e.g., a month) and entropy over a specified time period (e.g., a day), are indications of walking steadiness. In some embodiments, the gait compensatory model fuses inertial sensor signals (e.g., acceleration, rotation rate) to derive metrics/features for step length, walking speed and cadence, evaluate regularity and unpredictability (entropy) of step-to-step variation in each metric/feature over a specified time period (e.g., a day), determine metric/feature correlations for a user over the course of a specified time period (e.g., a month) and compute a degree of dispersions of the metric/feature correlations. Some example features include but are not limited to: variation of steps cadence speed within bouts and across several bouts, which can be calculated as an entropy value or using a chaotic deterministic system model, which can give a notion of step-to-step changes.

a. Entropy Metric

In some embodiments, an entropy metric output by the gait compensatory model is computed as follows.

For a given time-series of data: u(1), u(2), . . . u(N):

Entropy=Φ^{m}(r)−Φ^{m+1}(r), [1]

where for vectors x(i) composed of consecutive elements u(i):

x(1),x(2), . . . ,x(N−m+1) [2]

x(i)=[u(i),u(i+1), . . . ,u(i+m−1)], [3]

where Φ is defined as:

$${Φ}^{m} ( r ) = \frac{1}{N - m + 1} {\sum }_{i = 1}^{N - m + 1} log ( {C}_{i}^{m} ( r ) ) , and \qquad [ 4 ]$$

$${C}_{i}^{m} ( r ) = \frac{sum ( x ( j ) d [ x ( i ) , x ( j ) ] \leq r )}{N - m + 1} , and \qquad [ 5 ]$$

N is the number of elements in the time-series, m is the length of compared data (e.g., m=2. Note: step-to-step variance is most relevant to Kepler outcomes), and r is the filtering level (filtering level is defined as the modified discrete cosine (MDC) of each metric).

b. Degree of Dispersion Metric

FIG. 1B illustrates identifying compensatory walking patterns using a Poincare analysis. The top plot shows step length (m) versus time and the bottom plot shows step length N+1(m) versus step length N(m). An example cloud of data points are shown for an unsteady user bout (69 points, 5 falls) and a steady user (59 data points, 0 falls). The second plot is an ellipse-fitted Poincare plot. The dispersion of points perpendicular to the line-of-identity indicates the level of short-term variability. The dispersion of points along the line-of-identity indicates the level of long-term variability. Metrics that can be extracted from the ellipse-fitted Poincare plot include but are not limited to: a first standard deviation (SD1) along the width of the ellipse, which is a measure of short-term variation and a second standard deviation (SD2) along the length of the ellipse, which is a measure of long-term variation. Other metrics that can be extracted from the plot include the semi-minor axis and semi-major axis of the ellipse and the area of the ellipse.

iii. Anomalous Gait Model

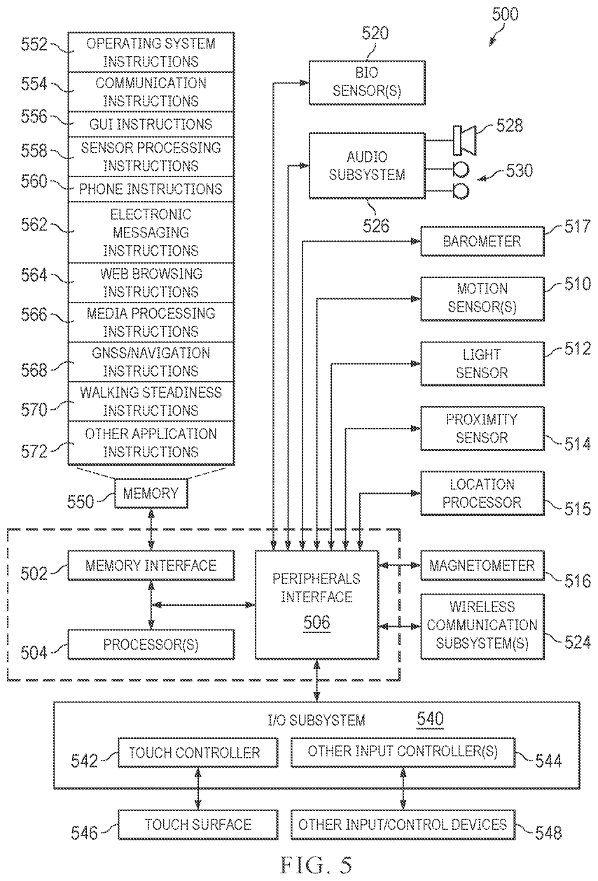

Typical walking is symmetric and can be characterized by a well-defined relationship of ground contact time (GCTs) (the amount of time feet are in contact with the ground). Deviations from this relationship are hard to achieve without significant declines in mobility, thus indicating anomalous gait. In some embodiments, asymmetry can be consistently observed over 0% (e.g., 10% or above) across many bouts. DST is longer (more time with both feet on ground), especially at slower speeds. The user's speed walking down stairs requires agility coordination and navigation, i.e., long times can be slow. In some embodiments, altitude data (e.g., barometer data) and acceleration data are fused to determine analogous gait. For example, a barometric pressure sensor (e.g., barometric sensor 517 in FIG. 5) or other sensor that can be used to estimate height or altitude to determine if the user is walking up/down stairs. The acceleration data is used to estimate the user's speed up/down the stairs.

iv. Gait Smoothness Model

In some embodiment, the gait smoothness component model determines, in the frequency domain, a closeness to a sine wave at the dominant walking frequency of the user, which is a marker of gait smoothness or stability.

FIG. 1C illustrates multi-directional gait smoothness, according to some embodiments. The top plot shows the frequency spectrum of anterior/posterior acceleration for a steady user and the bottom plot shows the frequency spectrum of anterior/posterior acceleration for an unsteady user. As can be observed in the frequency spectrum for an unsteady user, there are more peaks spread across the spectrum rather than a single peak at the fundamental walking frequency observed in the frequency spectrum of the steady user.

In some embodiments, gait smoothness can be calculated by:

$$Smoothness = \frac{Harmoni {c}_{dominant}^{2}}{{\sum }_{i = 1}^{10} Harmoni {c}_{i}^{2}} . \qquad [ 6 ]$$

where Harmonicᵢ is an index of harmonicity and Harmonic_{dominant} is the dominant harmonic.

Equation [6] calculates, for each direction separately (automatically detecting user's forward, back, side-to-side motion direction), gait smoothness by measuring closeness of the harmonics to sine waves in the dominant frequency.

v. Demographics/Null Model

In some embodiments, demographics/null model 113 provides a baseline notion of the user's capacity for their age based on, for example, a “soft” maximum (e.g., 95^{th} percentile) of bouts observed over a long window (e.g., 28 days). The features output by demographic/null model 113 include but are not limited to at least one of: age, height, BMI and speed Nth percentile (e.g., 90^{th} percentile).

vi. Behavioral Model

Characteristics of user behavior can either be informative of steadiness or a confound for reliably estimating steadiness over time. In some embodiments, a behavioral model is included in system 100 and configured to receive activity indicators and output indicators of behavior or changes in behavior that impact on walking steadiness. Some example behaviors include but are not limited to: variability in mobile device usage, variability in walking duration, changes in activity level, activity level, patterns of activity, prior detected falls, etc. Table I below summarizes the underlying premise of each behavior and the impact each behavior has on walking steadiness.

TABLE I
User Behavior Impact on Walking Steadiness

| Behavior | Underlying Premise | Impact on Steadiness |
|---|---|---|
| Variability | Estimation of steadiness | The disclose embodiments automatically |
| in device | requires an observation of | detect when a user is carrying the |
| usage | the user as they walk. A | device in the desired position. This |
| | user may or may not decide | includes fusing inertial sensor signals |
| | to bring their device with | (e.g., acceleration, rotation rate, |
| | them on said walks | pedometer step count) and implementing |
| | | models to compare sensor signals to |
| | | expectations for the known mechanics of |
| | | walking. When the sensor signals agree |
| | | with mechanical expectations, steadiness |
| | | is estimated. |
| Variability | For how long a user walks | Component models that do not take into |
| in walking | and the quality of the | account walking duration are necessary |
| duration | walking during a particular | to boost availability when particular |
| | duration can be informative | walk durations are low. The fusion model |
| | of steadiness. A user may | disclosed herein accounts for the varying |
| | decide to walk for a | availability of the underlying component |
| | particular duration for a | models due to changes in behavior of the |
| | variety of reasons (e.g. | user over time |
| | doing an intentional | |
| | walking workout, walking | |
| | to the mailbox, etc.). | |
| | The availability of certain | |
| | duration walks will be | |
| | variable depending on the | |
| | activities of the user as | |
| | well as how those | |
| | activities change over time | |
| Changes in | Dips in activity level of | Walking steadiness is evaluated over short |
| activity | the user can be related to | and long time scales to account for |
| level | dips in walking steadiness | infrequent and short-term deviations from |
| | (e.g. for acute injury), but | long-term expected behavior of the user. |
| | may also be entirely driven | A history is built for the user to |
| | by short-term changes in | determine whether a deviation is expected |
| | behavior of the user (e.g., | or a sign of an unexpected, potentially |
| | the user is on vacation). | concerning event. |
| Activity | Confirmed level of activity | With corroborating evidence, high activity |
| level | of the user provides an | levels of the user indicate good walking |
| | inferential signal of | steadiness. |
| | functional capacity. | |
| Patterns of | Episodic cues (e.g. workout | Provides context cues to interpret quality |
| activity | activity type and duration) | of walking steadiness estimates. Also, |
| | can distinguish confounds | provides evidence of good walking steadiness. |
| | like fatigue and activity- | |
| | related gait variations | |
| | from mobility limited causes. | |
| Prior | Confirmed evidence of past | With corroborating evidence, can indicate |
| detected | falls experienced by the | increased fall risk for the user. |
| falls | user has been shown to be | |
| | indicative of future fall | |
| | risk for the user (for | |
| | multiple fallers). | |

### IX. Fused Inference Model

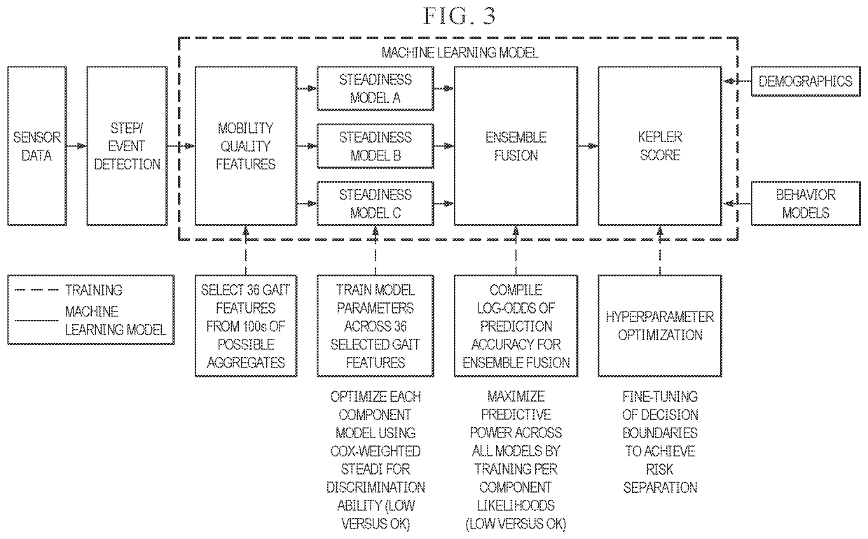

Fused inference model 105 infers an overall steadiness core 108 from the walking steadiness indicators output by component models 104. In an embodiment, fused inference model 105 includes an ensemble of machine learning steadiness models 304 (See FIG. 3) that are trained for their predictive discrimination power, with some ensemble models 304 being trained to fill in gaps in prediction from other ensemble models 304. The ensemble models 304 can be evaluated for their relative predictive power of fall risk, and the likelihoods of these evaluations can be used to form steadiness score 108 across all ensemble models 304 using, for example, a Bayesian inference formulation or other suitable statistical formulation.

There are multiple objectives in the estimation of walking steadiness, for which explicit tradeoffs may be specified, including correctly distinguishing curated examples of steady and unsteady behavior, reporting lower walking steadiness in cases where a user has self-identified mobility risk factors, and separating high and low walking steadiness users with a statistically verifiable distinction in fall hazard.

One or more of the ensemble models 304 can be constructed to capture different phenotypes of walking quality or corroborate one another by capturing similar phenotypes in different ways. The ensemble models 304 can have some diversity in their training to focus on different components of walking steadiness, but together form the ensemble that is combined into walking steadiness indicator 108. In an embodiment, the training of the ensemble models 304 includes forming outcome scores based on recorded user clinical data, such as surveys, as training targets for fall risk and other objectives.

The ensemble approach for fused inference model 105 described above provides several practical advantages. The approach allows for incremental improvements of the fall risk assessment at multiple levels. Its piecewise structure makes it easy to analyze and understand how it performs and why it works, and the limits of its applicability. The number of ensemble models 304 can range from the trivial case of a single model to any number of ensemble models. Thus, the ensemble approach also provides a forward-looking path to incorporate future improvements to system 100.

### X. Training Data

In some embodiments, the training of the models described above can be implemented in three steps: 1) training feature weights for component models, 2) performing maximum likelihood estimation from ensemble machine learning (ML) models, and 3) determining classification thresholds for the steadiness indicator/score. Training feature weights includes optimizing component models using weighted STEADI (Stopping Elderly Accidents, Deaths and Injuries) questions for discrimination ability (e.g., low risk versus high risk) that are consistent predictors of mobility limitations. Maximum likelihood estimation from the ensemble ML models maximizes predictive power across all models by training per component likelihoods (e.g., low risk versus high risk). Determining classification thresholds for the steadiness indicator/score includes determining additional cut-off resolution (e.g., low risk versus high risk).

i. Sources of Truth Data

To bring steadiness and fall risk together, in some embodiments training data for component models 104 include but are not limited to clinical assessments (e.g., STEADI questions) and fall data collected by mobile devices. Clinical assessments can include STEADI questionnaires that are weighted (e.g., weights from a Cox Proportionality Hazard Model) rather than assessing solely with measured outcomes. In some embodiments, modifications to the standard STEADI questionnaire that add or remove questions may be used (e.g., a mobility STEADI version that removes questions that are not consistent predictors of mobility limitations).

ii. Steadiness and Falls Together

In some embodiments, by mapping a small number of falls to a large number of STEADI questions, sufficient statistical power is gained for fall risk, thus accounting for the low confidence in quality of falls data reported, i.e., falls are notoriously incorrectly reported with poor time scales.

iii. Steadiness by Fall Type

In some embodiments, mapping STEADI questions to falls for a fall risk label, age and behavioral differences are accounted for by, e.g., excluding confounding data, such as sports falls. Additionally, a different Cox-model weight for questionnaire questions is created by age bucket for a smooth user experience (e.g., no discontinuities). In some embodiments, the fall data collected from mobile devices of users measures durations without fall. If a fall is recorded, the type of fall is recorded into a fall risk label along with the user's age and gender and date of fall. The fall data can be correlated with the user's responses to survey questionnaires of mobility risk factors (e.g., mobSTEADI), and built into a Cox proportionality model of fall risk that individually quantifies each factor in terms of its implied hazard ratio to falls as well as the p-value of the evidence.

The modelling process in the above step can be applied to cohorts of types of users (e.g., age range) with certain categories of falls to compute the combined sensitivities of risk factors with both age and behavior. The training of the models can then be conditioned in different ways, and specific models can be constructed to capture falls for joint age-behavior types in a manner that is supported by the evidence.

iv. Training of Feature Weights for Component Models—Basic Approach

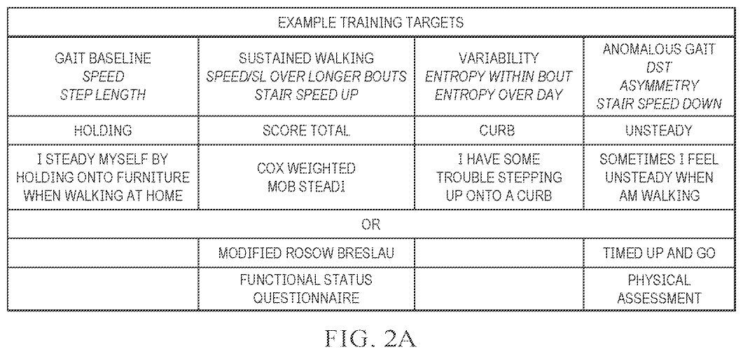

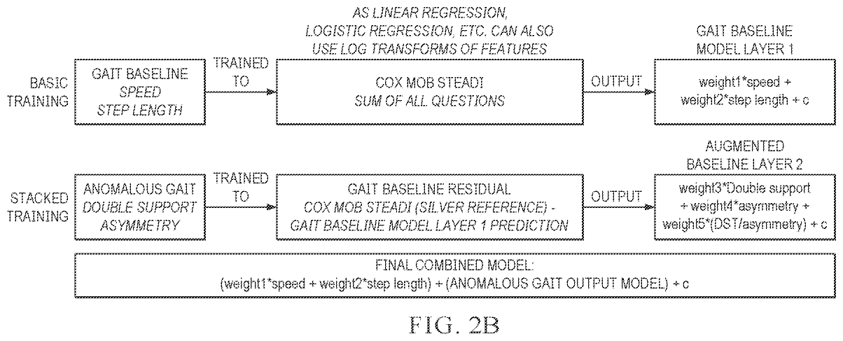

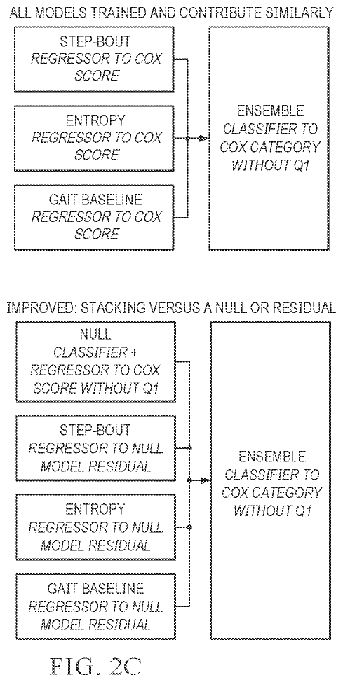

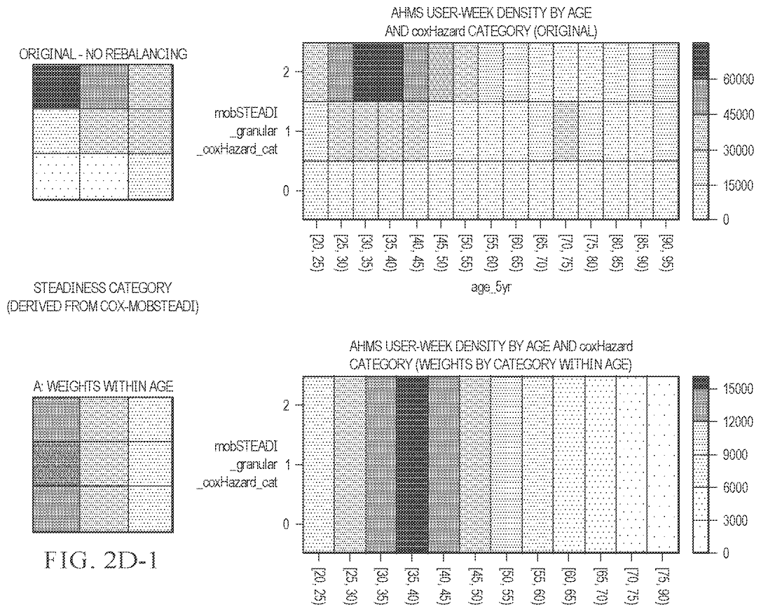

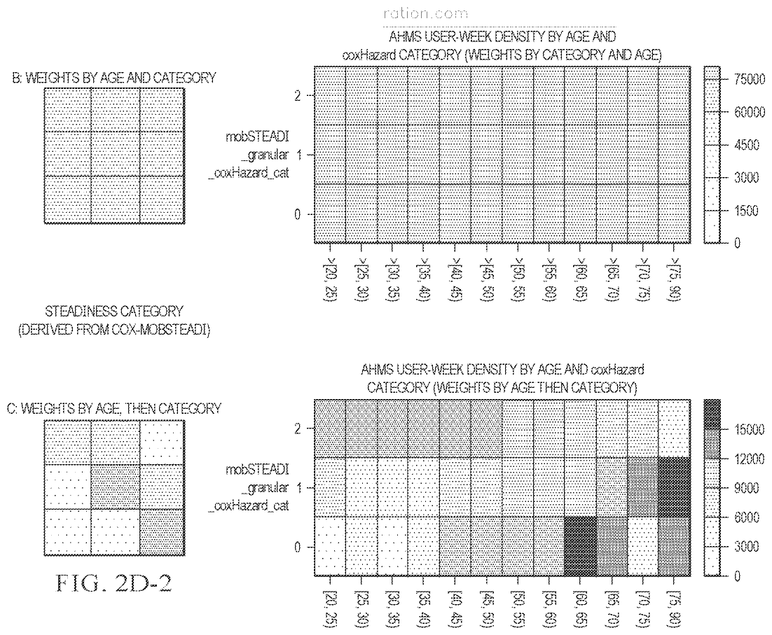

FIG. 2A illustrates a basic approach to training feature weights of component models, according to some embodiments. To capture the full signal in the fall risk label (e.g., Cox-weighted mobSTEADI), different models can be trained to different targets as illustrated in FIG. 2A. For example, different models can be trained to single STEADI questions, or the steadiness indicator 108 can be weighted in different ways. In some embodiments, if available, component models can be trained to a different proxy than a questionnaire, such as a TUG assessment or Functional Gait Assessment (FGA). In some embodiments, component models that are predictive of user capacity can be trained to functional questionnaires like a modified Rosow Breslau questionnaire, where users report how well they can do challenging tasks.

Referring again to FIG. 2A, one gait baseline feature is trained to a single STEADI question: “I hold myself by holding onto furniture when walking at home.” A sustained walking feature is trained to a Cox-weighted mobSTEADI question or a modified Rosow Breslau question or functional status questionnaire. A variability feature is trained to single STEADI question: “I have some trouble stepping up onto a curb.” An anomalous gait feature is trained to the single STEADI question: “Sometimes I feel unsteady when I am walking” or a Timed Up and Go functional assessment or FGA.

v. Training of Feature Weights—Stacked Approach

FIG. 2B illustrates training feature weights for component models using “stacked” models to train one submodel with a target based on the residual error of another submodel, according to some embodiments. In some embodiments, in addition to the training to a set of targets, model performance can be enhanced by “stacking” training models. In this example, the gait baseline is trained to a Cox-weighted STEADI questions (e.g., the sum of all questions) to provide a first model layer, e.g., weight1*speed+weight2*step_length+c, and the anomalous gait feature is trained to the gait baseline residual, e.g., weight3*DST+weight4*asymmetry+weight5*(DST/asymmetry)+c to provide an augmented baseline model layer, arriving at a final combined model is, e.g., (weight1*speed+weight2*step_length)+(anomalous gait output model)+c. In some embodiments, a stacking approach can be extended to training a number of models against the residual “null” model (i.e., basic model), as shown illustrated in FIG. 2C.

As shown in FIG. 2C, the top flow diagram shows a first approach where all models are trained and contribute similarly. As noted above, the stacked mode embodiments can be extended to train a residual against many models versus a null model (the most basic model possible) to only train for gains. In the top flow diagram, all models are trained and contribute similarly. For example, step-bout regressor to Cox score, entropy regressor to Cox score model and gait baseline regressor to Cox score are used to train the ensemble classifier to Cox Category without Q1.

The bottom flow diagram shows a second approach of stacking versus a null or residual model. Null classifier plus regressor to Cox score without Q1, step-bout regressor to null model residual, entropy regressor to nul model residual and gait baseline regressor to null model residual is used to train the ensemble classifier to Cox category without Q1.

vi. Training of Feature Weights—Age Balancing Approach

In some embodiments, the component models may have enhanced value if trained on different data subsets, e.g., 1) age restricted subsets (e.g., 40-65) to get better coverage/range of capacity for sustained walking features versus 65+ for anomalous gait, 2) with upweighting or down-weighting certain participants who are more trusted (e.g., fallers, or verified with independent mobility assessment), or 3) with age plus steadiness upweighting or down-weighting. An example is shown in FIGS. 2D-1 and 2D-2 of how age-steadiness relationships can be changed by upweight and down weights

vii. Training of Feature Weights—Activity Changes

Sudden changes in activity due to larger environmental or non-mobility related reasons can confound training, or make it more robust (e.g., help some models trained with sufficient data to learn aggressor scenarios). For models intended to capture unsteadiness during sustained walking behavior, the population-wide drops in activity (e.g., from forest fire, stay at home orders, etc.) can be kept to enhance their robustness. However, for other models that have interplays between activity and gait, the suppressed activity can be removed versus unsurpassed activity periods to ensure robustness. If there is a sudden activity change towards a sustained low activity period for a previously high user, the steadiness indicator 108 can be withheld or the amount of gait bouts increased before making a classification decision. In some embodiments, withholding can be overridden and “low risk” can still be called if sufficiently strong cues are observed, such as strong walking asymmetry value.

### XI. Training Ensemble—ML Architecture

FIG. 3 illustrates a training architecture 300 using an ensemble of machine learning models, according to some embodiments. The dashed arrows indicate the flow of training data and the solid arrows define the signal flow within the machine learning model. Sensor data 301 (e.g., acceleration, rotation rate) is input into step/event detector 302 (e.g., pedometer), which generates mobility quality features 303. During training, a subset of mobility quality features is selected to reduce specificity on the ensemble models 304 (e.g., steadiness model A, steadiness model B, steadiness model C, etc.).

Parameters of ensemble models 304 are trained across N (N=36) selected gait features to optimize each model using Cox-weighted STEADI, or other suitable approaches as described above, to obtain a scalar value with suitable discrimination ability (separating low risk versus OK risk). During training, outputs of ensemble models 304 are input into ensemble fusion model 305, which compiles log-odds of prediction accuracy for ensemble fusion 305, which maximizes predictive power across all ensemble models by training per model likelihoods (e.g., low versus OK). The output of ensemble fusion 305 is used in the Kepler score 306 assessment. During training, hyperparameter optimization is used to fine-tune decision boundaries in of Kepler score 306 to achieve risk separation based on demographics data 307 and behavior models 308.

### XII. Fusing Models

In some embodiments, consensus across ensemble models 304 can provide overall improvements in performance by increasing sensitivity in cases that one model confidently determines a case of instability missed by other models, or alternately combining partial evidence from a variety of models to build confidence in prediction of steadiness. Component model likelihoods can be based on age based priors, e.g., individual models may need to be weighted differently against each other for different age groups, e.g., <40 years. 40-65 and 65+ or even more granular (5-15 year age buckets).

In some embodiments, ensemble models 304 can be fused and weights can be obtained in a number of ways, e.g., odds ratio, mean log likelihood, voting scheme across likelihoods, a max, or specific logic rules (e.g., decision tree) where known model concordance or discordance can better predict falls or steadiness classifications.

i. Loss Function Options

In some embodiments, the loss function can upweight or down weight performance measures according to a desired end user experience (e.g., weight fall enrichment more heavily versus mobility risk factors, or weight specificity versus sensitivity more). In some embodiments, the fused data is given by:

$$fusion data = [ log \frac{p ( O K \vert {m}_{1} )}{p ( \sim OK \vert {m}_{1} )} , log \frac{p ( O K \vert {m}_{2} )}{p ( \sim OK \vert {m}_{2} )} , … log \frac{p ( O K \vert {m}_{n} )}{p ( \sim OK \vert {m}_{n )}} ] , \qquad [ 7 ]$$

where the likelihoods p(OK|m) and p(˜OK|m) are set by the definitions:
OK, notOK based on Cox-weighted mobSTEADI
OK, notOK based on survey results
OK, notOK based on functional assessments
OK, notOK based on demographic priors.
Empirical estimate of OK, notOK weighting based on representatives of data in dataset, and projected OKness for different user experiences.

The fusion data of Equation [7] can be composed into a loss function in a number of ways including but not limited to: Bayesian evidence of accumulation, mean log odds (similar to ensemble boosting), (signed) max log odds (normalized log likelihoods) and logical decision functions.

### Example Processes

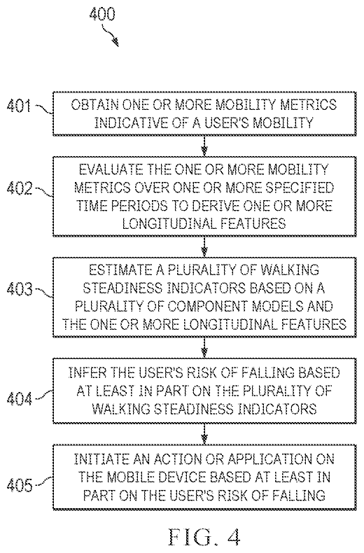

FIG. 4 is a flow diagram of process 400 for assessing walking steadiness and fall risk, according to some embodiments. Process 400 can be implemented by, for example, using system architecture 400 described in reference to FIG. 4.

Process 400 includes: obtaining one or more mobility metrics indicative of a user's mobility (401). The mobility metrics are obtained at least in part from sensor data output by at least one sensor of the mobile device.

Process 400 continues by evaluating the one or more mobility metrics over one or more specified time periods to derive one or more longitudinal features (402); estimating a plurality of walking steadiness indicators based on a plurality of component models and the one or more longitudinal features (403); inferring the user's risk of falling based at least in part on the plurality of walking steadiness indicators (404); and initiating an action or application on the mobile device based at least in part on the user's risk of falling (405). Each of these steps were described in detail in reference to FIGS. 1-3.

### Exemplary System Architectures

FIG. 5 illustrates example system architecture 500 implementing the features and operations described in reference to FIGS. 1-4. Architecture 500 can include memory interface 502, one or more hardware data processors, image processors and/or processors 504 and peripherals interface 506. Memory interface 502, one or more processors 504 and/or peripherals interface 506 can be separate components or can be integrated in one or more integrated circuits. System architecture 500 can be included in any suitable electronic device, including but not limited to: a smartphone, smartwatch, tablet computer, fitness band, laptop computer and the like.

Sensors, devices and subsystems can be coupled to peripherals interface 506 to provide multiple functionalities. For example, one or more motion sensors 510, light sensor 512 and proximity sensor 514 can be coupled to peripherals interface 506 to facilitate motion sensing (e.g., acceleration, rotation rates), lighting and proximity functions of the mobile device. Location processor 515 can be connected to peripherals interface 506 to provide geo-positioning. In some implementations, location processor 515 can be a GNSS receiver, such as the Global Positioning System (GPS) receiver. Electronic magnetometer 516 (e.g., an integrated circuit chip) can also be connected to peripherals interface 506 to provide data that can be used to determine the direction of magnetic North. Electronic magnetometer 516 can provide data to an electronic compass application. Motion sensor(s) 510 can include one or more accelerometers and/or gyros configured to determine change of speed and direction of movement. Barometer 517 can be configured to measure atmospheric pressure, which can be used to determine elevation changes. Biosensors 520 can include a heart rate sensor, such as a photoplethysmography (PPG) sensor, electrocardiography (ECG) sensor, etc.

Communication functions can be facilitated through wireless communication subsystems 524, which can include radio frequency (RF) receivers and transmitters (or transceivers) and/or optical (e.g., infrared) receivers and transmitters. The specific design and implementation of the communication subsystem 524 can depend on the communication network(s) over which a mobile device is intended to operate. For example, architecture 500 can include communication subsystems 524 designed to operate over a GSM network, a GPRS network, an EDGE network, a Wi-Fi™ network and a Bluetooth™ network. In particular, the wireless communication subsystems 524 can include hosting protocols, such that the mobile device can be configured as a base station for other wireless devices.

Audio subsystem 526 can be coupled to a speaker 528 and a microphone 530 to facilitate voice-enabled functions, such as voice recognition, voice replication, digital recording and telephony functions. Audio subsystem 526 can be configured to receive voice commands from the user.

I/O subsystem 540 can include touch surface controller 542 and/or other input controller(s) 544. Touch surface controller 542 can be coupled to a touch surface 546. Touch surface 546 and touch surface controller 542 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 546. Touch surface 546 can include, for example, a touch screen or the digital crown of a smart watch. I/O subsystem 540 can include a haptic engine or device for providing haptic feedback (e.g., vibration) in response to commands from one or more processors 504. In an embodiment, touch surface 546 can be a pressure-sensitive surface.

Other input controller(s) 544 can be coupled to other input/control devices 548, such as one or more buttons, rocker switches, thumb-wheel, infrared port and USB port. The one or more buttons (not shown) can include an up/down button for volume control of speaker 528 and/or microphone 540. Touch surface 546 or other controllers 544 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 546; and a pressing of the button for a second duration that is longer than the first duration may turn power to the mobile device on or off. The user may be able to customize a functionality of one or more of the buttons. The touch surface 546 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 502 can be coupled to memory 550. Memory 550 can include high-speed random access memory and/or non-volatile memory, such as one or more magnetic disk storage devices, one or more optical storage devices and/or flash memory (e.g., NAND, NOR). Memory 550 can store operating system 552, such as the iOS operating system developed by Apple Inc. of Cupertino, Calif. Operating system 552 may include instructions for handling basic system services and for performing hardware dependent tasks. In some implementations, operating system 552 can include a kernel (e.g., UNIX kernel).

Memory 550 may also store communication instructions 554 to facilitate communicating with one or more additional devices, one or more computers and/or one or more servers, such as, for example, instructions for implementing a software stack for wired or wireless communications with other devices, such as a sleep/wake tracking device. Memory 550 may include graphical user interface instructions 556 to facilitate graphic user interface processing; sensor processing instructions 558 to facilitate sensor-related processing and functions; phone instructions 560 to facilitate phone-related processes and functions; electronic messaging instructions 562 to facilitate electronic-messaging related processes and functions; web browsing instructions 564 to facilitate web browsing-related processes and functions; media processing instructions 566 to facilitate media processing-related processes and functions; GNSS/Location instructions 568 to facilitate generic GNSS and location-related processes and instructions; and walking steadiness instructions 570 that implement the features and processes described in reference to FIGS. 1-4. Memory 550 further includes other application instructions 572 for performing various functions using, for example, the fall risk estimate previously described (e.g., health monitoring or fitness applications).

Each of the above identified instructions and applications can correspond to a set of instructions for performing one or more functions described above. These instructions need not be implemented as separate software programs, procedures, or modules. Memory 550 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, with at least one processor of a mobile device, one or more mobility metrics indicative of a user's mobility, the mobility metrics obtained at least in part from a time series of sensor data output by at least one sensor of the mobile device;
evaluating, with the at least one processor, the one or more mobility metrics over one or more specified time periods to derive one or more longitudinal features indicative of variability of the user's gait; and
generating, with the at least one processor, at least one walking steadiness indicator for the user based on a gait compensatory model and the one or more longitudinal features.

2. The method of claim 1, wherein the at least one walking steadiness indicator is an entropy value computed over the one or more specified time periods.

3. The method of claim 1, wherein the at least one walking steadiness indicator is a degree of dispersion computed over the one or more specified time periods, the dispersion value computed by:
determining, with the at least one processor, metrics for step length, walking speed and cadence of the user based on the sensor data;
determining, with the at least one processor, correlations between the metrics over the one or more specified time periods; and
determining, with the at least one processor, the degree of dispersion based on the determined correlations.

4. The method of claim 3, wherein the degree of dispersion is determined based on parameters of an ellipse-fitted Poincaré plot that indicate at least one of short-term or long-term variability in the user's gait.

5. The method of claim 1, further comprising:
determining, with the at least one processor and prior to the evaluating, that the sensor data covers a duration that satisfies a minimum threshold time.

6. The method of claim 1, further comprising:
generating, with the at least one processor, the at least one walking steadiness indicator based on the gait compensatory model, the one or more longitudinal features and population norms.

7. The method of claim 6, wherein the population norms are represented as percentiles by age.

8. A method comprising:
obtaining, with at least one processor of a mobile device, one or more mobility metrics indicative of a user's mobility, the mobility metrics obtained at least in part from a time series of sensor data output by at least one sensor of the mobile device;
evaluating, with the at least one processor, the one or more mobility metrics over one or more specified time periods to derive one or more longitudinal features indicative of sustained walking; and
generating, with the at least one processor, at least one walking steadiness indicator for the user based on a capacity model and the one or more longitudinal features.

9. The method of claim 8, wherein generating the at least one walking steadiness indicator based on the capacity model and the one or more longitudinal features, further comprises:
estimating vertical acceleration from the sensor data;
estimated walking speed from the vertical acceleration;
estimating cadence and step length of the user for each step cycle based on the sensor data;
generating the at least one walking steadiness indicator for the user based on the estimates of user walking speed, step length and cadence.

10. The method of claim 8, wherein generating the at least one the walking steadiness indicator based on the capacity model and the one or more longitudinal features, further comprises:
estimating periods of continuous stepping based on the sensor data;
detecting periods of consecutive stepping based on the sensor data;
grouping the periods into time periods of specified durations; and
evaluating features for particular time periods to generate the at least one walking steadiness indicator.

11. The method of claim 10, wherein the features include at least one of: cadence, step length, double step time, median speed over a specified time duration or speed coefficient of variance.

12. The method of claim 8, further comprising:
generating, with the at least one processor, the at least one walking steadiness indicator based on the capacity model, the one or more longitudinal features and population norms.

13. The method of claim 12, wherein the population norms are represented as percentiles by age.

14. A method comprising:
obtaining, with at least one processor of a mobile device, one or more mobility metrics indicative of a user's mobility, the mobility metrics obtained at least in part from a time series of sensor data output by at least one sensor of the mobile device;
evaluating, with the at least one processor, the one or more mobility metrics over one or more specified time periods to derive one or more longitudinal features indicative of frequency characteristics of the user's walking pattern; and
generating, with the at least one processor, at least one walking steadiness indicator for the user based on a gait smoothness model and the one or more longitudinal features.

15. The method of claim 14, wherein the gait smoothness model calculates gait smoothness using an index of harmonicity to determine closeness of the user's gait to a sine wave at a dominant frequency of the user's walking.

16. The method of claim 14, wherein the smoothness is calculated for multiple directions of the user's motion.

17. The method of claim 14, further comprising:
generating, with the at least one processor, the at least one walking steadiness indicator based on the gait smoothness model, the one or more longitudinal features and population norms.

18. The method of claim 17, wherein the population norms are represented as percentiles by age.

19. A method comprising:
obtaining, with at least one processor of a mobile device, training data indicative of mobility limitations; and
training, with the at least one processor and using the training data, a plurality of machine learning models for estimating walking steadiness, wherein the plurality of component models are trained to different targets.

20. The method of claim 19, wherein training data includes weighted questions for fall risk discrimination that are consistent predictors of mobility limitations.

21. The method of claim 20, wherein the questions are Cox-weighted.

22. The method of claim 3, wherein different Cox-weights are based on age.

23. The method of claim 19, wherein the training data includes at least one of functional gait assessments or functional status questionnaires.

24. The method of claim 19, wherein training the plurality of machine learning models for estimating walking steadiness includes training the machine learning models to their own residuals.

25. The method of claim 24, wherein the residuals are trained against a plurality of the machine learning models versus a null model.

26. The method of claim 24, wherein training the plurality of machine learning models includes training the plurality of machine learning models to the residuals of other submodels.
